(19) United States
(12) Patent Application Publication
(10) Pub. No.: US 2010/0268323 A1
(43) Pub. Date: Oct. 21, 2010

(54) Inflammation Accelerating Prosthesis

(76) Inventor: Daniel Sullivan, Medina, MN (US)

Correspondence Address: INSKEEP INTELLECTUAL PROPERTY GROUP, INC, 2281 W. 190TH STREET, SUITE 200, TORRANCE, CA, 90504, US

(21) Appl. No.: 12/303,934

(22) PCT Filed: Jun. 8, 2007
(86) PCT No.: PCT/US07/70800

§ 371 Date: Jun. 23, 2010

Related U.S. Application Data
(60) Provisional application No. 60/812,251, filed on Jun. 8, 2006.

(51) Int. Cl. A61F 2/82; A61F 2/84

(52) U.S. Cl. 623/1.15; 623/1.23

(57) ABSTRACT

A prosthesis sleeve that is deployed within a patient's vessel at a damaged tissue area. The sleeve includes an inflammatory inducing section that releases materials near the damaged tissue to accelerate or enhance the immune response for a period of time sufficient to result in a smooth layer of repaired tissue.

### RELATED APPLICATIONS

This application claims priority to International Patent Application No. PCT/US2007/070800, International Filing Date Jun. 8, 2007, entitled Inflammation Accelerating Prosthesis, and to U.S. Provisional Application Ser. No. 60/812,251 filed Jun. 8, 2006 entitled Inflammation Accelerating Prosthesis, both of which are hereby incorporated by reference.

### BACKGROUND OF THE INVENTION

Injury to a circulatory structure of a patient (e.g., a blood vessel) due to physical injury (e.g. stent placement or angioplasty procedures) or disease (e.g., atherosclerosis or infection) typically results in an immunologic response to contain, repair and eventually heal the injured tissue. However, in some circumstances this immune healing response can permanently damage the vessels by producing unwanted scar tissue, re-narrowing of an artery (e.g., restenosis) or hardening of the vessels.

For example, often angioplasty permanently opens previously occluded blood vessels; however, restenosis, thrombosis, or vessel collapse may occur following angioplasty. Restenosis refers to the re-narrowing of an artery after an initially successful angioplasty due to exaggerated healing which causes a proliferation of tissue in the angioplasty area. Thrombosis is a clotting within a blood vessel which may cause infection to tissues supplied by the blood vessel. In order to prevent restenosis and vessel collapse, stents of various configurations have been used to hold the lumen of a blood vessel open following angioplasty. However, stents do not entirely reduce the occurrence of thrombotic abrupt closure due to clotting; stents with rough surfaces exposed to blood flow may actually increase thrombosis, and restenosis may still occur because tissue may grow through and around the lattice of the stent.

Accordingly, in an effort to treat such undesirable effects and/or conditions, many current treatment techniques have been developed and are presently used which focus on reducing or eliminating these vessel complications by delivering anti-inflammatory materials (e.g., biologically active agents) to an inflamed region of a vessel. Some techniques include applying an anti-inflammatory compound on a stent and positioning the stent within a diseased region of a vessel, allowing the anti-inflammatory compounds to migrate into the tissue. Examples of coated stents can be found in U.S. Pat. No. 6,071,305, which describes directional delivery of a biologically active agent (e.g., anti-inflammatory, as disclosed at column 5, line 8) via a non-biodegradable, expandable stent, wherein the directional delivery occurs via at least one fluid opening in only a portion of the stent by diffusion, in a manner sufficient to “prevent inflammation” (column 4, lines 63-65).

Additional examples of drug-delivery stents are described U.S. Pat. No. 7,214,383 (the '383 patent), as well as U.S. Patent Publication Nos. 20060099236 and 20060035879 (the '236 Publication and '879 Publication, respectively). The '383 patent describes a stent having a drug composition deposited on the outer lumen surface, which is stated to “reduce inflammatory responses from the body of the patient receiving the stent” (column 1, lines 27-28). Both the '236 Publication and the '879 publication describe stents that have anti-inflammatory properties. Some examples of anti-inflammatory compounds and their delivery can be seen in U.S. Pat. Nos. 6,232,297; 6,514,949; and 6,537,977 and U.S. Applications Nos. 20060099236; 20060035879; 20050278929; 20050055078; 20040225346; 20040219147; 20040039438; and 20010007083; the contents of which are hereby incorporated by reference.

U.S. Pat. Nos. 6,232,297; 6,514,949; and 6,537,977 (the '297 patent, the '949 patent and the '977 patent, respectively) describe anti-inflammatory compounds and methods that inhibit the effects of or otherwise prevent the mammalian inflammatory response in various ways. The '297 patent describes methods and compounds used for inhibiting the inflammatory response. The '949 patent describes methods and compounds used to protect tissues from the effects of the inflammatory response. The '977 patent describes the use of an agent that enhances the anti-inflammatory effect.

In each of the above-referenced patents and publications, the importance of preventing or slowing the inflammatory response via application of anti-inflammatory compounds and/or methods are described and addressed. However, in some circumstances, anti-inflammatory compounds can in fact prolong the overall time of the immune response from the patient's body, leading to long term damage and related complications.

In view of these possible complications, alternative treatment methods are needed to reduce or eliminate inflammatory-related complications within a patient's circulatory system.

### BRIEF DESCRIPTION OF THE DRAWINGS

FIG. 1 illustrates a preferred embodiment of an inflammation generating sleeve according to the present invention;

FIG. 2 illustrates the inflammation generating sleeve seen in FIG. 1; and

FIG. 3 illustrates another preferred embodiment of an inflammation generating sleeve positioned over a stent according to the present invention.

### DETAILED DESCRIPTION OF THE INVENTION

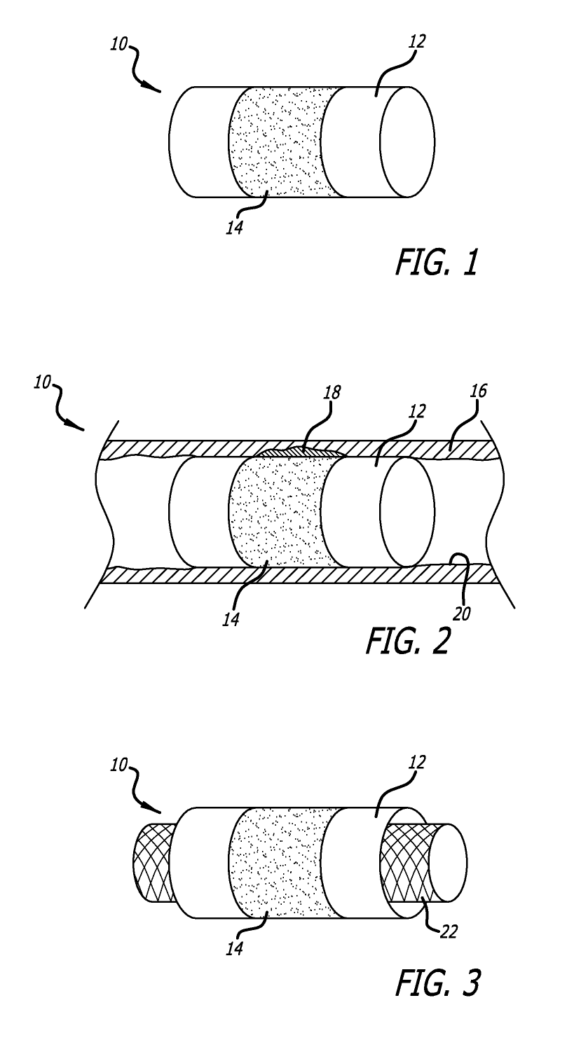

Referring to FIG. 1, one embodiment according to the present invention provides a prosthesis 10 having a sleeve 12 and an inflammatory portion 14. The sleeve 12 may act as merely a substrate for the inflammatory portion 14 or may have sufficient structure to apply outward pressure to the inside surface of the inflammatory portion so as to push the inflammatory portion 14 against a lumen wall in a stent-like fashion. The sleeve 12 is preferably constructed from bioabsorbable or biodegradable material, known in the art, such that the sleeve 12 dissolves and is absorbed into the body after the healing response is completed. One preferred example of bioabsorbable material would include a bioabsorbable synthetic polymer. Using a bioabsorbable or biodegradable material limits the impact of the implant and prevents the implant from causing an undesired thrombotic response. The material should be selected and sized to remain in place and exert pressure (if applicable) for a predetermined amount of time according to the application. Factors to consider include the size of the area being treated, the size of the blood vessel, and the amount of flow through the blood vessel. Higher flow typically results in a faster degradation rate. Alternatively, the sleeve 12 may be constructed of a nondegradable material known in the art, such as metals (e.g., Nitinol mesh), if the situation warrants a permanent implant.

Similarly, the inflammatory portion 14 of the prosthesis 10 is an area containing a bioreactive material, such as a drug, chemical or protein. Almost any material can be used in the inflammatory portion 14 that generates an inflammatory response. Preferably inflammatory materials are selected that easily pass into the nearby tissue of the lumen wall. The inflammatory portion 14 may comprise a bioreactive material coated onto a substrate or the inflammatory portion 14 or simply be an area of the sleeve 12 onto which a bioreactive material is applied. For example, if the sleeve 12 is to constructed of a nondegradable material, it may be desired to utilize a inflammatory portion 14 with a biodegradable substrate. It may also simplify manufacturing to produce a sheet of material with a substrate and a bioreactive agent applied thereto for forming individual inflammatory of various shapes or sizes, rather than “painting” various shapes on sleeves 12 using the bioreactive agents.

Whether the inflammatory portion 14 uses the sleeve 12 as a substrate or contains includes its own substrate, it may be made from any biodegradable or bioabsorbable materials that eventually disperse leaving a smooth, healed internal lumen wall 20. Any material such as a drug, chemical or protein can be coated, impregnated, or otherwise stored within the inflammatory portion 14 of the sleeve 12 so as to appropriately release into or around the damaged portion.

Turning now to FIG. 2, the prosthesis 10 is shown implanted within a body lumen 16 at or near a damaged portion 18. The inflammatory portion 14 is preferably positioned at least partially over the area of the lumen wall 20 containing the damaged portion 18. The inflammatory portion 14 of the prosthesis 10 can be formulated to recognize the body's natural response to injury. It may also be formulated to release an inflammatory material that enhances and accelerates the inflammatory process. Exemplary inflammatory materials are known to those of skill in the art and may include the substances identified in U.S. Publication No. 2006/0116666 entitled Two-Stage Scar Generation for Treating Atrial Fibrillation, the entire contents of which are hereby incorporated by reference. Thus, the damaged portion 18 will be more quickly healed and permanent damage can be limited, thereby creating a smooth, healed lumen wall 20. Further, the subsequent immune response may even contain the damaged portion 18, further limiting long term damage to the lumen wall. Because the inflammatory portion can be sized and shaped for any specific individual application, the accelerated healing response is confined to a discrete region and the damaged portion 18 heals rapidly.

As seen in FIG. 3, the prosthesis 10 is preferably positioned around a stent 22 (either self expanding or balloon expandable) which presses the sleeve 12 of the prosthesis 10 against the inner surface of the lumen wall when both are deployed. In this respect, the stent 22 provides an anchoring force to maintain the location of prosthesis 10 within the lumen 16. Preferred embodiments of stents contemplated for use in the present invention would include helically-wound wire (e.g., single-stranded, twisted or braided multi-stranded) and welded wires, from metal or plastics including biodegradable plastics such as polylactic acid, shape memory effect materials, superelastic materials and polymers.

Alternatively, the prosthesis 10 may be deployed alone, without a stent 22. In this respect, it is preferred that the prosthesis 10 be self-expanding (e.g., composed of a shape memory material such as Nitinol) or mechanically expandable, such as balloon-expandable.

Alternatively, the inflammatory portion 14 of the prosthesis 10 alone can be positioned over a stent 22 or even incorporated into the structure or coating of a stent 22.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A prosthesis comprising:
a tubular member having an expanded shape and a compressed shape; said tubular member having a segment configured to generate an inflammatory response in an adjacent tissue.

2. The prosthesis of claim 1 wherein said tubular member comprises a stent.

3. The prosthesis of claim 1 wherein said tubular member further comprises a sleeve disposed around the outer surface of said tubular member.

4. The prosthesis of claim 1 wherein said segment comprises a medicament-containing portion.

5. The prosthesis of claim 1 wherein said tubular member comprises a biodegradable material.

6. The prosthesis of claim 1 wherein said tubular member comprises a memory metal.

7. The prosthesis of claim 1 wherein said segment comprises a bioreactive agent disposed on a substrate.

8. The prosthesis of claim 7 wherein said substrate comprises a biodegradable material.

9. The prosthesis of claim 1 wherein said segment comprises a bioreactive agent disposed directly on said tubular member.

10. The prosthesis of claim 1 wherein said tubular member having an expanded shape comprises said tubular member having an expanded shape sufficient to place it is direct contact with adjacent tissue.

11. A prosthesis capable of generating a tissue response in a body lumen comprising:
a sleeve;
an inflammatory portion disposed on an outside surface of said sleeve, said inflammatory portion sized and shaped for therapeutic interaction with a damaged portion of said body lumen.

12. The prosthesis of claim 11 wherein said sleeve comprises a biodegradable material.

13. The prosthesis of claim 11 wherein said sleeve is suited for placement around an outside surface of a stent.

14. The prosthesis of claim 11 wherein said sleeve is expandable such that, in an expanded configuration, said sleeve presses said inflammatory portion against an inner wall of said body lumen, proximate said damaged portion.

15. A method of accelerating a healing response in a wounded portion of tissue in a body lumen comprising the steps of:
introducing a prosthesis comprising an inflammatory portion into said body lumen; and
positioning said prosthesis within said lumen such that said inflammatory portion contacts said damaged portion of tissue;
wherein said contact of said inflammatory portion with said wounded tissue portion promotes healing of said damaged portion of tissue.

16. The method of claim 15 wherein introducing a prosthesis comprising an inflammatory portion into said body lumen comprises introducing a prosthesis comprising introducing a prosthesis comprising an inflammatory portion sized and shaped for treatment of said damaged portion of tissue.

17. The method of claim 15 wherein introducing a prosthesis comprising an inflammatory portion into said body lumen comprises introducing a biodegradable prosthesis comprising an inflammatory portion into said body lumen.

18. The method of claim 15 wherein positioning said prosthesis within said lumen such a that said inflammatory portion contact said damaged portion of tissue comprises positioning said prosthesis within said lumen such that said inflammatory portion contact said damaged portion of tissue for a predetermined period of time.

19. The method of claim 18 wherein said predetermined period of time is controlled by constructing said inflammatory portion using biodegradable materials sized and shaped to decompose after said predetermined period of time.

20. The method of claim 15 wherein introducing a prosthesis comprising an inflammatory portion into said body lumen comprises placing said prosthesis around a stent and introducing the stent into the body lumen.
